(19) United States
(12) Patent Application Publication
(10) Pub. No.: US 2007/0254338 A1
(43) Pub. Date: Nov. 1, 2007

(54) Method for making recombinant protein using complementation dependent DHFR mutants

(75) Inventors: R. Guy Caspary, Seattle, WA (US); Allison Bianchi, Seattle, WA (US); Pauline S. Smidt, Seattle, WA (US); Virginia L. Price, Seattle, WA (US); Martin J. Allen, Seattle, WA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMGEN INC., Thousand Oaks, CA (US)

(21) Appl. No.: 11/789,339

(22) Filed: Apr. 24, 2007

Related U.S. Application Data
(60) Provisional application No. 60/794,337, filed on Apr. 24, 2006.

(51) Int. Cl. C12P 21/06; C12N 9/06; C07H 21/04; C12N 15/74; C12N 1/21

(52) U.S. Cl. 435/69.1; 435/191; 435/252.3; 435/471; 536/23.2

(57) ABSTRACT

The present invention relates to compositions and methods for making recombinant heteromeric proteins using a protein complementation assay employing complementation pairs of selectable markers.

The present application claims the priority benefit of U.S. Provisional Patent Application No. 60/794,337, filed Apr. 24, 2006, incorporated herein be reference.

### FIELD OF THE INVENTION

The present invention relates generally to the field of recombinant expression of polypeptides in animal cell culture. More particularly, the invention provides compositions and methods for recombinant expression of heteromeric proteins using a protein complementation assay employing selectable markers.

### BACKGROUND OF THE INVENTION

Many commercially important proteins are produced in recombinantly engineered cells that have been adapted for long term growth in culture. Frequently, the recombinant proteins are expressed as a single polypeptide chain, even if the protein comprises multiple subunits. Alternatively, multiple heterologous polypeptides that associate to form heteromeric complexes, such as for example, an antibody, which is formed by the expression and association of equal parts of heavy chains and light chains, are expressed as single subunits which associate in the cytoplasm after expression.

Protein complementation assays have been developed for studying protein-protein interaction and heteromeric protein or protein complex assembly in vitro. Several variations of protein complementation assays have been reported. For example, a ubiquitin-based split protein sensor (USPS) (Johnsson et al., Proc. Natl. Acad. Sci. USA 91:10340-44, 1994) has been developed, and is based on cleavage of proteins with N-terminal fusions to ubiquitin by cytosolic proteases (ubiquitinases) that recognize its tertiary structure. The strategy depends on the reassembly of the tertiary structure of the ubiquitin protein from complementary N- and C-terminal fragments and crucially, on the augmention of this reassembly by oligomerization domains fused to these fragments. Reassembly as allows for specific proteolysis of the assembled product by cytosolic proteases (ubiquitinases). Fusion of a reporter protein-ubiquitin C-terminal fragment could also be cleaved by ubiquitinases, but only if co-expressed with an N-terminal fragment of ubiquitin that complements the C-terminal fragment. The reconstitution of observable ubiquitinase activity only occurs if the N- and C-terminal fragments are bound through GCN4 leucine zippers (O'Shea et al., Science 254:539-44, 1991), Ellenberger et al., Cell 71:1223-37, 1992).

Rossi, et al. (Proc. Nat. Acad. Sci. USA 94:8405-10, 1997) reported an assay based on the classical complementation of α and ω fragments of β-galactosidase (β-gal) and induction of complementation by inducing oligomerization of the proteins FKBP12 and theits target rapamycin in transfected C2C12 myoblast cell lines. Reconstitution of β-gal activity is detected using substrate fluorescein di-β-D-galactopyranoside using several fluorecence detection assays. Krevolin et al. (U.S. Pat. No. 5,362,625) taught the use of this complementation assay to detect protein-protein interactions. Also β-gal complementation in mammalian cells has previously been reported (Moosmann et al., Nucl. Acids Res. 24:1171-72, 1996). Other assays useful to detect protein interaction include yeast two hybrid assays (Vojtek et al., Cell. 74:205-214, 1993) and yeast split hybrid assays (Shih et al., Proc Natl Acad Sci USA. 93:13896-901, 1996).

One difficulty that can be encountered when expressing heteromeric complexes in cells is obtaining appropriate amounts of each of the recombinant polypeptides that forms a component of the complex. For example, in the expression of an antibodies either the heavy chain or the light chain is frequently expressed at relatively high levels with respect to the corresponding partner; however, obtaining a cell line expressing both chains at high levels and in roughly equal amounts is difficult. As a result, in mammalian cells, an antibody heavy chain is often not secreted in the absence of light chain (Struzenberger et al., J. Biotechnol. 69:215-226, 1999). These difficulties result in additional steps and also repetition of steps in the process of generating cell lines expressing recombinant polypeptides resulting in delays which substantially increase costs associated with recombinant expression of the polypeptides.

Thus there remains a need in the art to provide improved methods for selecting cells expressing recombinant polypeptides and for expressing heteromeric polypeptides in appropriate ratios for optimal association and large scale production in cell culture.

### SUMMARY OF THE INVENTION

The present invention provides compositions and methods for making recombinant heteromeric proteins using modified selectable marker sequences, wherein a functional selectable marker is detected only in the presence of the complementation pair members.

In one aspect, the invention provides a first isolated nucleic acid molecule comprising a sequence encoding a first polypeptide, wherein the first polypeptide is a subunit of a heteromeric protein, and a sequence encoding a first complementation pair member of a full-length selectable marker, wherein said first complementation pair member comprises a first amino acid mutation or modification that reduces selectable marker activity of the first complementation pair member such that selectable marker activity can be observed only in the presence of a second complementation pair member of the selectable marker which complements the first modification.

In a related aspect, the invention provides a second isolated nucleic acid molecule comprising a sequence encoding a second polypeptide, wherein the second polypeptide is a subunit of a heteromeric protein, and a sequence encoding a second complementation pair member of a full-length selectable marker, wherein said second complementation pair member comprises a second amino acid mutation or modification that reduces selectable marker activity of the second complementation pair member such that selectable marker activity can be observed only in the presence of a first complementation pair member of the selectable marker which complements the second modification.

In another aspect, the second nucleic acid is the invention provides a second isolated nucleic acid molecule comprising: a sequence encoding a second polypeptide, wherein the second polypeptide is a subunit of a heteromeric protein, wherein the heteromeric protein is a heteromeric protein comprising the first polypeptide, and a sequence encoding a second complementation pair member of a full-length selectable marker, wherein the selectable marker is the same selectable marker of the first complementation pair member of a full-length selectable marker, wherein said second complementation pair member of a selectable marker comprises a second amino acid mutation or modification that reduces selectable marker activity of the second complementation pair member such that selectable marker activity can be observed only in the presence of the first complementation pair member of the selectable marker.

In a further aspect, the invention provides an expression vector comprising the first nucleic acid. In a related embodiment, an expression vector comprising the second nucleic acid is contemplated. In a further embodiment, it is contemplated that the expression vector comprising the first nucleic acid further comprises the second nucleic acid.

It is contemplated that the first polypeptide and second polypeptides of the invention each encode a subunit of a heteromeric protein. In one embodiment, the first polypeptide comprises an antibody light chain or an antigen binding fragment thereof. In a related embodiment, the second polypeptide comprises an antibody heavy chain or a antigen binding fragment thereof.

In one aspect, the invention contemplates that the nucleic acid comprises a selectable marker selected from the group consisting of a drug resistance marker, a metabolic survival marker, a color marker and a fluorescent marker.

In another aspect, the invention provides isolated first and second nucleic acid molecules wherein the complementation pair members of a selectable marker are the same in the first nucleic acid and the second nucleic acid. In one embodiment, the selectable marker of complementation pair members of a selectable marker, is selected from the group consisting of dihydrofolate reductase, neomycin resistance, hygromycin resistance, beta-galactosidase, and green fluorescent protein. In a preferred embodiment, the selectable marker is a DHFR gene.

In one aspect the complementation pair members comprises one or more amino acid mutations or modifications in the DHFR molecule. In a related aspect, the first nucleic acid comprises a first amino acid mutation or modification. In another aspect, the second nucleic acid comprises a second amino acid mutation or modification. In one embodiment, the first amino acid mutation or modification is in a Fragment 1, 2 region of the DHFR selectable marker. In a related aspect, the first amino acid mutation or modification is selected from the group consisting of a mutation at glutamic acid 30 of mouse DHFR and a rigid peptide linker inserted between Fragment 1, 2 and Fragment 3 of DHFR. In a further aspect, the first amino acid mutation or modification is Glu30Ala.

In still yet another aspect, the first mutation or modification is a rigid peptide linker which prevents proper folding of the selectable marker protein, selected from the group consisting of an oligoproline sequence, an oligoglycine sequence, Gly-Gly-Pro repeats, GGGGS (SEQ ID NO: 1) repeats and the amino acid sequence PDALEAEIARLRKQIEALQGQNQHLQAAISQLKKVELFP (SEQ ID NO: 2). In one embodiment, the rigid peptide linker comprises the amino acid sequence GGPGGP (SEQ ID NO: 3). In a related embodiment, the rigid peptide linker comprises the amino acid sequence PDALEAEIARLRKQIEALQGQNQHLQAAISQLKKVELFP (SEQ ID NO: 2).

In another aspect, it is contemplated that the first amino acid mutation or modification is in a Fragment 3 subunit of the DHFR marker. It is contemplated that the first amino acid mutation or modification is a mutation at glycine 116 of mouse DHFR. Exemplary mutations include Gly116Ala.

In a related aspect, the second amino acid mutation or modification is in a Fragment 1, 2 region of the DHFR marker. It is contemplated that the second amino acid mutation or modification is selected from the group consisting of a mutation at glutamic acid 30 in mouse DHFR, and a rigid peptide linker inserted between Fragment 1, 2 and Fragment 3 of DHFR. In one embodiment, the second amino acid mutation or modification is Glu30Ala.

In a further embodiment, the second amino acid mutation or modification is a rigid peptide linker selected from the group consisting of an oligoproline sequence, an oligoglycine sequence, Gly-Gly-Pro repeats, GGGGS repeats and the amino acid sequence PDALEAEIARLRKQIEALQGQNQHLQAAISQLKKVELFP (SEQ ID NO: 2). It is contemplated that the rigid peptide linker comprises the amino acid sequence GGPGGP. It is further contemplated that the rigid peptide linker is the amino acid sequence PDALEAEIARLRKQIEALQGQNQHLQAAISQLKKVELFP.

In a further aspect, the second amino acid mutation or modification is in a Fragment 3 subunit of the DHFR marker. It is contemplated that the second amino acid mutation or modification is a mutation at glycine 116 of mouse DHFR In one embodiment, the second amino acid mutation or modification is Gly116Ala.

The invention further provides isolated nucleic acid molecules of the invention wherein the first or second complementation pair member of a selectable marker is a fusion polypeptide comprising an interaction domain. In one aspect the interaction domain is capable of directing multimerization of multitude of subunits. Interaction domains contemplated by the invention include dimerization domains, trimerization domains, tetramerization domains, and the like. In one embodiment, the interaction domain is a leucine zipper from a polypeptide selected from the group consisting of GCN4, C/EBP, c-Fos, c-Jun, c-Myc and c-Max.

The invention further provides that the isolated nucleic acids of the invention further encode a different functional selectable marker selected from the group consisting of zeomycin, neomycin, puromycin, Blasticidin S, and GPT.

The invention further provides a host cell comprising the isolated nucleic acid molecules of the invention. In one aspect, the first and second nucleic acids are expressed on separate vectors in the host cell. In a related aspect, the first and second nucleic acids are expressed on the same vector in the host cell. The invention contemplates that the host cell is selected from the group consisting of CHO, VERO, BHK, HeLa, Cos, MDCK, 293, 3T3, a myeloma cell line, and WI38 cells.

The invention also contemplates a method of recombinantly expressing a heteromeric polypeptide comprising culturing the host cell comprising the first and second nucleic acids under conditions wherein the heteromeric protein is expressed. It is contemplated that the method further comprises isolating the heteromeric protein. It is also contemplated that the host cell useful in the method of the invention is selected from the group consisting of CHO, VERO, BHK, HeLa, Cos, MDCK, 293, 3T3, a myeloma cell line, and WI38 cells.

In one aspect, the heteromeric protein is an antibody.

### DETAILED DESCRIPTION OF THE INVENTION

The present invention relates to novel vectors and methods useful for the production of recombinant heteromeric proteins, wherein the subunits of the proteins are expressed in molar ratios beneficial for association in vitro leading to increased protein yield.

The invention utilizes two or more copies of a selectable marker expressed in the same host cell, each of which has one or more mutations or modifications in one or more locations of the protein sequence such that no single copy of the mutated selectable marker has significant activity, i.e., activity is reduced or even non-existent. Mutations or modifications include but are not limited to, amino acid substitution, deletion, insertion or any other modification useful to alter the activity of a protein such as alteration of amino acid sidechain properties, glycosylation, and other modifications known in the art. The mutations or modification in each protein sequence are located in distinct locations in each copy of the selectable marker protein such that when all copies are expressed in the same cell and they associate, the individual copies of the selectable marker are able to complement, i.e., overcome, the inactivating mutations, thereby providing a selectable activity. The complementary copies of the selectable marker are referred to herein as “complementation members.”Detectable activity of the complementation members depends upon their interaction, which, in certain aspects, can be facilitated by interaction domains. Such interaction domains can be endogenous to the complementation pair member or it can be heterologous to the complementation pair member.

Thus, the term “complementation member” as used herein refers to two or more nucleotide or amino acid sequences which each encode the same protein, wherein each member comprises a mutation or modification that inactivates or reduces the functional activity of the protein itself. When each complementation member is expressed in the same cell, the mutations or modifications in one copy compensates for the mutations or modifications in another copy, thereby forming a functional protein. In aspects wherein complementation is effected through the interaction of two mutated or modified copies of the selectable marker protein, the two copies are referred to as “complementation pair members.”

In one aspect, the invention entails the use of two complementary pair members of a selectable marker, each expressed as a fusion protein with an interaction domain. When expressed in this way, the interaction domains promote association or dimerization of the complementary pair members thereby allowing the inactive (or reduced activity) molecules to form an active protein and providing a selectable activity. It is contemplated that the interaction domain is a dimerization domain, a trimerization domain, a tetramerization domain, or any domain involved in multimerizing a protein. Exemplary interaction domains include, but are not limited to, leucine zipper domains, helix-loop-helix domains, and ultimerization domains found in the E. coli lactose repressor.

The term “transformed” or “transfected” as used herein refers to a host cell modified to contain an exogenous polynucleotide, which can be integrated into the chromosome of the host cell or maintained as an episomal element. It is contemplated that in certain aspects of the methods provided, the host cell is transfected in a “transfection step.” The method may comprise multiple transfection steps. In addition, other methods known in the art for introducing exogenous polynucleotides into a host cell, including for example, electroporation and cell fusion which are not technically “transformation” are within the definition of the term “transformation for purposes of this description.

The term “heteromeric complex” as used herein refers to a molecular complex formed by the association of at least two different molecules. The association can be non-covalent interaction or covalent attachment, e.g., disulfide bonds. The two different molecules are typically two different polypeptides, however, the invention contemplates heteromeric complexes between polypeptides and nucleic acids. In one embodiment, the heteromeric complex provides a functional activity of the expressed copies of the proteins, such as the ability to bind a substrate (e.g., an immunoglobulin capable of binding a corresponding antigen), enzymatic activity or the like. In one embodiment, the heteromeric complex is secreted into the culture medium of the host cell in which it is being produced, and in other embodiments, the complex forms within the host cell in either the ctytoplasm or the nucleus.

The term “antibody” is used in the broadest sense and includes fully assembled antibodies, monoclonal antibodies, chimeric antibodies, human or humanized antibodies, multispecific antibodies (e.g., bispecific antibodies), a complementary determining region (CDR)-grafted antibody, antibody fragments that can bind antigen (e.g., Fv, Fab, Fab′, F′(ab)2) and recombinant peptides comprising the forgoing provided the antibody associates as a heteromeric complex.

As used herein, the term “fusion protein” refers to a protein, or domain of a protein (e.g., a soluble extracellular domain) fused to a heterologous protein or peptide. Any of the molecules herein described can be expressed as a fusion protein including but not limited to the extracellular domain of a cellular receptor molecule, an enzyme, a hormone, a cytokine, a portion of an immunoglobulin molecule, a zipper domain, and an epitope. Non-limiting examples of such fusion proteins include proteins expressed as a fusion with a portion of an immunoglobulin molecule, proteins expressed as fusion proteins with a zipper moiety, and novel polyfunctional proteins such as fusion proteins of cytokines and growth factors (i.e., GM-CSF and IL-3, MGF and IL-3). WO 93/08207 and WO 96/40918 describe the preparation of various soluble oligomeric forms of a molecule referred to as CD40L, including an immunoglobulin fusion protein and a zipper fusion protein, respectively; the techniques discussed therein are applicable to other proteins.

In one aspect the invention contemplates that the complementation members are linked to an interaction domain. An interaction domain is a protein domain that can join with at least one other protein domain and facilitate multimerization of the proteins to which they are linked. Exemplary interaction domains include, but are not limited to, dimerization domains, trimerization domains, and tetramerization domains. In one embodiment the interaction domain is a leucine zipper coiled coil polypeptide. A leucine zipper typically comprises about 35 amino acids containing a characteristic seven residue repeat with hydrophobic residues at the first and fourth residues of the repeat (Harbury et al., Science 262:1401-7, 1993). Thus, a leucine zipper is amenable to fusion to a polypeptide for the purpose of oligomerizing the polypeptide as it is a small protein molecule and is less likely to disrupt the polypeptides normal function than would a larger interaction domain. Examples of leucine zippers include but are not limited leucine zipper domains from polypeptides such as GCN4, C/EBP, c-Fos, c-Jun, c-Myc and c-Max.

In another embodiment the interaction domain is a dimerization domain. A dimerization domain can be a polypeptide capable of inducing interaction or association of two polypeptides. There are two types of dimers, those capable of forming homodimers (with the same sequence), or heterodimers (with another sequence). Examples of dimerization domains include, but are not limited to, helix-loop-helix domains (Murre et al., Cell 58:537-544, 1989), for example in, the retinoic acid receptor, thyroid hormone receptor, other nuclear hormone receptors (Kurokawa et al., Genes Dev. 7:1423-35, 1993) and yeast transcription factors GAL4 and HAP1 (Marmonstein et al., Nature 356:408-414, 1992; Zhang et al., Proc. Natl. Acad. Sci. USA 90:2851-55, 1993; U.S. Pat. No. 5,624,818), which all have dimerization domains with a helix-loop-helix motif. Additional dimerization domains are known in the art.

In yet another embodiment, the interaction domain is a trimerization domain, which is a polypeptide capable of binding two other tetimerization domains to form a trimeric complex. Examples of proteins containing a trimerization domain include, but are not limited to, bacteriophage T4 fibritin (Meier et al., J Mol. Biol 344:1051-69, 2004) and NF-kappaB essential modulator (NEMO) (Agou et al., J Biol Chem. 279:27861-9, 2004.)

In a further embodiment, the interaction domain is a tetramerization domain, which is a polypeptide capable of binding three other tetramerization domains to form a tetrameric complex. Examples of proteins containing tetramerization domains include but are not limited to the E. coli lactose repressor (amino acids 46-360; Chakerian et al., J. Biol. Chem. 266:1371-4, 1991; Alberti et al., EMBO J. 12:3227-36, 1993; and Lewis et al., Nature 271:1247, 1996), and the p53 tetramerization domain at residues 322-355 (Clore et al., Science 265:386, 1994; Harbury et al., Science 262:1401, 1993; U.S. Pat. No. 5,573,925).

It is further contemplated that the interaction domain includes domains that allow multimerization of any number of subunits.

Selectable markers that confer resistance to particular drugs that are ordinarily toxic to an animal cell can be used in the methods and compositions of the invention. For example, the following are non-limiting examples of resistance selectable markers: zeomycin (zeo); puromycin (PAC); Blasticidin S (BlaS), GPT, which confers resistance to mycophenolic acid (Mulligan & Berg, Proc. Natl. Acad. Sci. USA 78:2072-6, 1981); the neomycin resistance gene, which confers resistance to the aminoglycoside G-418 (Colberre-Garapin et al., J. Mol. Biol. 150:1-14, 1981); and hygro (hph gene), which confers resistance to hygromycin (Santerre et al., Gene 30:147-56, 1984). Additional selectable markers are known in the art and useful in the compositions and methods of the invention.

Metabolic enzymes that confer cell survival or induce cell death under prescribed conditions can also be used in the methods and compositions of the inventions. Examples include, but are not limited to: dihydrofolate reductase (DHFR); herpes simplex virus thymidine kinase (TK) (Wigler et al., Cell 11:223-32, 1977), hypoxanthine-guanine phosphoribosyltransferase (HGPRT) (Szybalska & Szybalski, Proc. Natl. Acad. Sci. USA 48:2026-34, 1962), and adenine phosphoribosyltransferase (APRT) (Lowy et al., Cell 22:817-23, 1980), which are genes which can be employed in cells lacking TK, HGPRT or APRT, respectively.

In one embodiment, dihydrofolate reductase (DHFR) is the selectable marker used in the methods and compositions of the present invention. DHFR is involved in converting dihydrofolate into tetrahydrofolate, which is required for de novo synthesis of purines, thymidylic acid and certain amino acids. Several DHFR genes have been sequenced to date, including several bacterial DHFR, such as E. coli DHFR and Plasmodium DHFR, and mammalian DHFR, including murine DHFR (Genbank Accession No. NM_{—}010049), human DHFR (Genbank Accession No. NM_{—}000791), and hamster DHFR (Genbank Accession No. L15311).

Murine DHFR (mDHFR) shares high sequence identity with the human DHFR (hDHFR) sequence (91% identity) and is highly homologous to the E. coli enzyme (29% identity, 68% homology) and these sequences share considerable tertiary structure (Volz et al., J. Biol. Chem. 257:2528-36, 1982). Comparison of the crystal structures of mDHFR and hDHFR suggests that their active sites are essentially identical (Oefner et al., Eur. J. Biochem. 174, 377-85, 1988; Stammers, et al. FEBS Lett. 218, 178-84, 1987). DHFR has been described as being formed of three structural fragments forming two domains. [Gegg, et al., in Techniques in Protein Chemistry (eds. Marshak, D. R.) 439-448 (Academic Press, New York, USA, 1996); Bystroff et al., Biochem. 30:2227-39, 1991]. DHFR has been divided into the adenine binding domain (residues 47 to 105, fragment[2]) and a discontinuous domain (residues 1 to 46, fragment[1] and a third domain from residues 106 to 186 (fragment[3]), numbering according to the murine sequence. The folate binding pocket and the NADPH binding groove are formed mainly by residues belonging to fragments[1] and [2] (F[1,2]). Fragment [3] (F[3]) is not directly implicated in catalysis, but is necessary for function of the DHFR protein.

Residues 101 to 108 of hDHFR, at the junction between F[2] and F[3], form a disordered loop which lies on the same face of the protein as both termini. Studies have demonstrated that cleavage of mDHFR between F [1,2] and [3], at residue 107, minimizes disruption of the active site and NADPH cofactor binding sites. The native N-terminus of mDHFR and the novel N-terminus created by cleavage occur on the same surface of the enzyme (Oefner, et al., supra, Stammers et al, supra) allowing for ease of N-terminal covalent attachment of each fragment to associating fragments such as the leucine zippers or other interaction domains. Michnick (U.S. Pat. Nos. 6,270,964 and 6,929,916) and Pelletier et al. (Proc. Natl. Acad. Sci. USA 95, 12141-46, 1998) describe vectors comprising a first polypeptide linked to the DHFR F[1,2] subunit and a second polypeptide that binds to the first polypeptide, wherein the second polypeptide is linked to the DHFR F[3] fragment. Transfection of a single cell with the two DHFR fragment constructs (F[1,2]+F[3]) yields a fully active DHFR protein. These constructs were used to detect interaction of the first and second polypeptides in vitro or in vivo (Pelletier et al., supra, and U.S. Pat. No. 6,270,964).

Genetically engineered or naturally-occurring DHFR-deficient cell lines require glycine, a purine, (e.g., hypoxanthine), and thymidine (GHT media) for growth because these cells are unable to reduce folate supplied in the medium to the active form of cofactor, tetrahydrofolate, required for cell growth. Withdrawal of GHT from the medium (−GHT) requires that the cells then express a fully active DHFR gene for survival. As such, DHFR deficient cell lines transfected with active DHFR are useful tools for selecting cells transfected with a DHFR-containing plasmid of interest.

For recombinant protein production, cells lacking DHFR activity such that they will not grow in selection media (−GHT) without the DHFR activity may be transfected with DHFR fragment constructs as described in Pelletier (supra). Viability and growth of the cells is restored upon association of the DHFR fragments in vitro. Alternatively, DHFR transfection into cells is also useful for conferring antimetabolite resistance to methotrexate (Wigler et al., Proc. Natl. Acad. Sci. USA 77:3567-70, 1980; O'Hare et al., Proc. Natl. Acad. Sci. USA 78:1527-31, 1981). Methotrexate is a folic acid derivative that interferes with folic acid metabolism and s cytotoxic to cells. Cells expressing endogenous DHFR can be used and transfectants receiving additional DHFR copies can be selected by conferring increased resistance to toxic levels of methotrexate.

Methotrexate can also be used in accordance with the invention to amplify recombinant nucleic acids after selection of (−GHT) sensitive cells. Selection is commonly at a concentration of 25 nM, more preferably 50 nM, even more preferably 150 nM and most preferably 300 nM of methotrexate. The skilled artisan will recognize that methotrexate concentrations can be as high as 500 nM or higher to amplify recombinant nucleic acids that give resistance to the drug, such as those described herein. Amplification using the vectors and methods of the invention is particularly advantageous because it has been found that in the case of expressing a heavy and light chain, both chains are amplified in roughly equal levels.

As described herein, methods are provided utilizing full-length DHFR molecules which comprise an inactivating fragment in one of the F[1,2] or the F[3] subunits. The invention provides that when a full-length DHFR having an inactivating or activity reducing modification in the F[1,2] region (Fragment A) is co-expressed with a nucleotide sequence comprising a full-length DHFR gene having an inactivating or activity reducing modification in the F[3] region (Fragment B), the two modifications successfully complement each other and provide a fully functional DHFR molecule. In one embodiment the modification in the N-terminal fragment, Fragment A, is in the catalytic binding site. In another embodiment, the mutation is a change of the glutamic acid at residue 30 of mouse DHFR. In a related embodiment, the mutation is a change of the glutamic acid residue 30 to an alanine, Glu30Ala. In a further embodiment, the mutation in the C-terminal fragment, Fragment B, interferes with bonds important in the protein folding. For example, the invention contemplates a mutation of glycine 116 such that the mutation interferes with protein folding. In one embodiment the mutation at glycine 116 is glycine to alanine, Gly116Ala.

In another embodiment, the DHFR fragments do not include specific (or point) amino acid mutations that result in reduced activity, but instead are modified by insertion of a rigid linker sequence (RL) between the Fragment A and Fragment B regions. A rigid linker, also known as a molecular ruler, is a sequence of amino acids which are stearically hindered in their conformation such that they prevent the adjacent amino acids from moving in space and folding together. Separation of the DHFR F[1,2] and F[3] subunits by a rigid linker or another linker sequence interferes with correct peptide folding. Complementation of a full length DHFR protein comprising a rigid linker requires co-expression of a second full-length DHFR molecule also separated by a rigid linker and association of the two full length proteins which allows association of the subunits, for example, subunits F[1,2] on one full length molecule with F[3] on the other full length molecule. In one embodiment, the rigid linker is a rigid oligoproline linker. The linker may have 16-20 residues as described in the art (Arora et al., J Am Chem. Soc. 124:13067-71, 2002). In another embodiment the linker is a (GGGGS)_{N} linker. In related embodiment, the linker peptide comprises at least one gly-gly-pro (GGP) repeat. In still a further embodiment, the linker is an amino acid sequence which forms an extended alpha helical coiled coil structure. An exemplary sequence contemplated by the invention is PDALEAEIARLRKQIEALQGQNQHLQAAISQLKKVELFP (SEQ ID NO: 2).

Nucleotide sequences may be joined together using well-established recombinant DNA techniques (see Sambrook J et al. (2d Ed.; 1989) Molecular Cloning: A Laboratory Manual, Cold Spring Harbor Laboratory, Cold Spring Harbor, N.Y.). Useful nucleotide sequences for joining to polypeptides include multiple vectors, for example, plasmids, cosmids, lambda phage derivatives, phagemids, and the like, that are well-known in the art. In general, the vector contains an origin of replication functional in at least one organism, convenient restriction endonuclease sites, and a selectable marker for the host cell which is different than the selectable marker of the complementation pairs.

Vectors according to the invention include expression vectors, replication vectors, probe generation vectors, sequencing vectors, and retroviral vectors. Vectors contemplated by the invention include, but are not limited to, microorganisms such as bacteria transformed with recombinant bacteriophage, plasmid, phagemid, or cosmid DNA expression vectors; yeast transformed with yeast expression vectors; insect cell systems infected with viral expression vectors (e.g., baculovirus); plant cell systems transfected with virus expression vectors (e.g., Cauliflower Mosaic Virus, CaMV; Tobacco Mosaic Virus, TMV) or transformed with bacterial expression vectors (e.g., Ti or pBR322 plasmid); or even animal cell systems.

Mammalian expression vectors typically comprise an origin of replication, a suitable promoter, and also any necessary ribosome binding sites, polyadenylation site, splice donor and acceptor sites, transcriptional termination sequences, and 5′ flanking nontranscribed sequences. DNA sequences derived from the SV40 viral genome, for example, the SV40 origin, early promoter, enhancer, splice, and polyadenylation sites may be used to provide the required expression control elements. Exemplary eukaryotic vectors include pcDNA3, pWLneo, pSV2cat, pOG44, PXTI, pSG (Stratagene) pSVK3, pBPV, pMSG, pSVL, and pVITRO3.

Accordingly, the invention also provides a vector including a polynucleotide of the invention and a host cell containing the polynucleotide. A host cell according to the invention can be a prokaryotic or eukaryotic cell and can be a unicellular organism or part of a multicellular organism. Any host/vector system can be used to express one or more of the polynucleotides encoding polypeptides useful in the present invention. Mature proteins can be expressed in mammalian cells, yeast, bacteria, or other cells under the control of appropriate promoters. Appropriate cloning and expression vectors for use with prokaryotic and eukaryotic hosts are described by Sambrook, et al., in Molecular Cloning: A Laboratory Manual, 2^{nd} Edition, Cold Spring Harbor, N.Y. (1989), the disclosure of which is hereby incorporated by reference. Mammalian cells that are useful in recombinant protein production include, but are not limited to, a myeloma cell line, VERO cells, HeLa cells, Chinese hamster ovary (CHO) cells, COS cells (such as COS-7), WI38, BHK, HepG2, 3T3, RIN, MDCK, A549, PC12, K562 and HEK 293 cells.

Examples of heteromeric complexes contemplated by the invention, in addition to immunoglobulins, include, but are not limited to, any heterodimeric or hetero-oligomeric protein, e.g., BMP2/BMP7, osteogenic protein, interleukin 1 converting enzyme (ICE), various interleukin receptors (e.g., the IL-18 receptor, IL-13 receptor, IL-4 receptor and IL-7 receptor), receptors of the nucleus such as retinoid receptors, T-cell receptors, integrins such as cell adhesion molecules, betal-integrins, tumor necrosis factor receptor and soluble and membrane bound forms of class I and class II major histocompatibility complex proteins (MHC). For heteromeric complexes that are receptors, the invention encompasses both soluble and membrane bound forms of the polypeptides. Descriptions of additional heteromeric proteins that can be produced according to the invention can be found in, for example, Human Cytokines. Handbook for Basic and Clinical Research, Vol. II (Aggarwal and Gutterman, eds. Blackwell Sciences, Cambridge Mass., 1998); Growth Factors: A Practical Approach (McKay and Leigh, Eds. Oxford University Press Inc., New York, 1993) and The Cytokine Handbook (A W Thompson, ed.; Academic Press, San Diego Calif.; 1991).

In one aspect, the heteromeric complex of the invention is an immunoglobulin molecule. The immunoglobulin in vertebrate systems is an antibody comprised of two identical light chains and two identical heavy chains. The four chains are joined together by disulfide bonds, such that each light chain is joined with a heavy chain and the heavy chains are connected across their tails altogether forming a Y-shaped heteromeric complex. Numerous techniques are known by which DNA encoding immunoglobulin molecules can be manipulated to yield DNAs capable of encoding recombinant proteins such as antibodies with enhanced affinity, or other antibody-based polypeptides (see, for example, Larrick et al., Biotechnology 7:934-38, 1989; Reichmann et al., Nature 332:323-27, 1988; Roberts et al., Nature 328:731-34, 1987; Verhoeyen et al., Science 239:1534-36, 1988; Chaudhary et al., Nature 339:394-97, 1989).

Antibody includes fully assembled antibodies, monoclonal antibodies, chimeric antibodies, human or humanized antibodies, multispecific antibodies (e.g., bispecific antibodies), a complementary determining region (CDR)-grafted antibody, antibody fragments that can bind antigen (e.g., Fv, Fab, Fab′, F′(ab)2) and recombinant peptides comprising the forgoing provided the antibody associates as a heteromeric complex.

An antibody that is specific for its antigen indicates that the variable regions of the antibodies of the invention recognize and bind the polypeptide of interest exclusively (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies of the invention can be produced using any method well known and routinely practiced in the art.

Monoclonal antibody refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Recombinant monoclonal antibodies to be used in accordance with the present invention may be made initially by the hybridoma method first described by Kohler et al. (Nature, 256:495 [1975), and sequenced for use in the present invention. The monoclonal antibodies may also be isolated from phage antibody libraries using the techniques described in Clackson et al. (Nature, 352:624-628, 1991) and Marks et al. (J. Mol. Biol., 222:581-597, 1991).

Chimeric monoclonal antibodies, in which the variable Ig domains of a mouse monoclonal antibody are fused to human constant Ig domains (See Morrison et al., Proc. Natl. Acad. Sci. USA 81, 6841-55, 1984); and, Boulianne et al, (Nature 312:643-46, 1984) are contemplated by the invention.

Non-human antibodies may be humanized by any methods known in the art. A preferred “humanized antibody” has a human constant region, while the variable region, or at least a CDR, of the antibody is derived from a non-human species. Methods for humanizing non-human antibodies are well known in the art. (see U.S. Pat. Nos. 5,585,089, and 5,693,762). Generally, a humanized antibody has one or more amino acid residues introduced into its framework region from a source which is non-human. Humanization can be performed, for example, using methods described in the art [e.g., Jones et al., Nature 321: 522-525, 1986; Riechmann et al., Nature, 332: 323-327, 1988; Verhoeyen et al., Science 239:1534-1536, 1988; and WO 93/11236], by substituting at least a portion of a rodent complementarity-determining region for the corresponding regions of a human antibody. Numerous techniques for preparing engineered antibodies are described the art [e.g., Owens et al., J. Immunol. Meth., 168:149-165, 1994]. Further changes can then be introduced into the antibody framework to modulate affinity or immunogenicity.

Rapid, large-scale recombinant methods for generating antibodies may be employed, such as phage display [Hoogenboom et al., J. Mol. Biol. 227:381-88, 1992; Marks et al., J. Mol. Biol. 222: 581-97, 1991] or ribosome display methods, optionally followed by affinity maturation [see, e.g., Ouwehand et al., Vox Sang 74(Suppl 2):223-232, 1998; Rader et al., Proc. Natl. Acad. Sci. USA 95:8910-8915, 1998; Dall'Acqua et al., Curr. Opin. Struct. Biol. 8:443-450, 1998]. Phage-display processes mimic immune selection through the display of antibody repertoires on the surface of filamentous bacteriophage, and subsequent selection of phage by their binding to an antigen of choice. One such technique is described in WO 99/10494, which describes the isolation of high affinity and functional agonistic antibodies for MPL and msk receptors using such an approach.

Antibodies having specificity for more than one antigen, including bispecific antibodies, trispecific antibodies, etc. are contemplated by the invention. Bispecific antibodies are monoclonal, preferably human or humanized, antibodies that have binding specificities for at least two different antigens. Bispecific antibodies have been produced, isolated, and tested using standard procedures described in the literature. See, e.g., Pluckthun & Pack, Immunotechnology, 3:83-105, 1997; Carter et al., J. Hematotherapy, 4: 463-470, 1995; Renner & Pfreundschuh, Immunological Reviews, 145:179-209, 1995; Segal et al., J. Hematotherapy, 4: 377-382, 1995; Segal et al., Immunobiology, 185: 390-402, 1992; and Bolhuis et al., Cancer Immunol. Immunother., 34: 1-8, 1991, and U.S. Pat. No. 5,643,759, all of which are incorporated herein by reference in their entireties.

Bispecific antibodies have also been generated via phage display screening methods using the so-called hierarchical dual combinatorial approach as disclosed in WO 92/01047 in which an individual colony containing either an H or L chain clone is used to infect a complete library of clones encoding the other chain (L or H) and the resulting two-chain specific binding member is selected in accordance with phage display techniques such as those described therein. This technique is also disclosed in Marks et al., (Bio/Technology 10:779-783, 1992). Heavy and light chain variable regions derived from an antibody library can be used in the method of the invention to formulate multispecific antibodies.

Recombinant cells producing fully human antibodies (such as are prepared using antibody libraries, and/or transgenic animals, and optionally further modified in vitro), as well as humanized antibodies can also be used in the invention. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger et al., WO 86/01533; Neuberger et al., European Patent No. 0,194,276 B1; Winter, U.S. Pat. No. 5,225,539; Winter, European Patent No. 0,239,400 B1; Queen et al., European Patent No. 0,451,216 B1; and Padlan et al., European Patent No. 0,519,596 A1. For example, the invention can be used to induce the expression of human and/or humanized antibodies that immunospecifically recognize specific cellular targets, including, but not limited to, the human EGF receptor, the her-2/neu antigen, the CEA antigen, Prostate Specific Membrane Antigen (PSMA), CD5, CD11a, CD18, NGF, CD20, CD45, Ep-cam, other cancer cell surface molecules, TNF-alpha, TGF-b 1, VEGF, other cytokines, alpha 4 beta 7 integrin, IgEs, viral proteins (for example, cytomegalovirus), etc.

As an additional aspect, the invention includes kits which comprise one or more isolated nucleic acids of the invention packaged in a manner which facilitates their use to practice methods of the invention. In one embodiment, such a kit includes a nucleic acid as described herein (e.g., a nucleic acids comprising complementation pairs of a selectable marker), packaged in a container such as a sealed bottle or vessel, with a label affixed to the container or included in the package that describes use of the compound or composition in practicing the method. In one embodiment, the nucleic acid of the invention is packaged in a unit dosage form. Preferably, the kit contains a label that describes use of the antibody composition.

Additional aspects and details of the invention will be apparent from the following examples, which are intended to be illustrative rather than limiting.

### EXAMPLE 1

### Generation of Modified DHFR Molecules

DHFR was reported to be divided into two distinct functional subunits, the F[1,2] fragment which comprises the DHFR catalytic activity and the F[3] fragment involved in protein folding. Using one of the DHFR subunits linked to a protein of interest and the other subunit linked to a second protein of interest, interaction between the two proteins could be detected [U.S. Pat. Nos. 6,270,964 and 6,929,916 and Pelletier et al. (Proc. Natl. Acad. Sci. USA 95, 12141-46, 1998].

In order to determine if full-length DHFR constructs exhibited similar complementary function, and could be used to express heteromeric proteins recombinantly, modified DHFR molecules were designed such that the modified DHFR molecules were inactive alone, but provided selectable marker activity when expressed in conjunction with a complementary construct.

The modified DHFR molecules were designed with the mutations or modifications described below and generated by Blue Heron Biotechnology (Bothell, Wash.) in pUC based vectors. The DHFR constructs were then cut out of the pUC vector using restriction sites placed at the end of the constructs and ligated into the pVITRO3 vector (InvivoGen, San Diego, Calif.). Transfection of the vectors comprising the modified DHFR molecules into DHFR deficient CHO cell line was performed using standard transfection protocols. Cells were incubated at 37° C. until in log phase, and transfected with an appropriate concentration of purified plasmid using electroporation settings optimized for CHO cells.

Initial selection was performed in shake flasks in non-DHFR selection media (+glycine, hypoxanthine and thymidine, GHT) plus hygromycin (250 μg/ml) with recovery of up to 90% recovery, followed by selection in DHFR selection media lacking glycine, hypoxanthine and thymidine (−GHT) with selection to 90% recovery.

In an initial experiment, the DHFR mutated and modified constructs were compared to controls and their effect and cell survival in the presence and absence of DHFR growth medium assessed. The mutation in Fragment A was a Glu30Ala mutation, designated as A*. The mutation in Fragment B, the C-terminal fragment, was a Gly116Ala mutation designated as B*. For those mutants containing the “RL” rigid linker the sequences of the linker was PDALEAEIARLRKQIEALQGQNQHLQAAISQLKKVELFP (SEQ ID NO: 2). Split DHFR (F[1,2]+F[3]) was used as a positive control for the assays.

Results of the assay demonstrated that in cells expressing complementation pairs [A-GGP-B*+B-A*] and [A-B*+A*-B] significant cell survival was regained 7 passages after culture with −GHT selection media. Complementation pair [A-RL-B+B-RL-A] demonstrated survival rates above that for split DHFR, exhibiting approximately 80% cell survival after culture with −GHT selection media, improving to approximately 100% survival after approximately 6 passages. Complementation pair [A-B*+A*-B] demonstrated survival rates moderately below split DHFR, but was similar to split DHFR and DHFR complementation pair [A-RL-B+B-RL-A] after 5 cell passages in selection media.

The modified DHFR single pair members were also assessed for the ability to sustain survival of transfected cells without a complementary pair member. Only the DHFR modified B-RL-A exhibited any survival of cells upon withdrawal of +GHT media, recovering up to approximately 65% cell survival after 8 passages in −GHT selection media. All other single complementation pair members were unable to sustain cell growth in DHFR selection media.

These results demonstrate that the DHFR complementation pairs are effective at conferring survival to cells containing both members of the pair. Further, the DHFR complementation pair members provide a useful method for expressing subunits of a heteromeric protein in a highly selectable environment such that the subunits are expressed at proportional levels.

Numerous modifications and variations in the invention as set forth in the above illustrative examples are expected to occur to those skilled in the art. Consequently only such limitations as appear in the appended claims should be placed on the invention.

What is claimed:

1. A first isolated nucleic acid molecule comprising a sequence encoding a first polypeptide, wherein the first polypeptide is a subunit of a heteromeric protein, and a sequence encoding a first complementation pair member of a full-length selectable marker, wherein said first complementation pair member comprises a first amino acid mutation or modification that reduces selectable marker activity of the first complementation pair member such that selectable marker activity can be observed only in the presence of a second complementation pair member of the selectable marker which complements the first modification.

2. A second isolated nucleic acid molecule comprising a sequence encoding a second polypeptide, wherein the second polypeptide is a subunit of a heteromeric protein, and a sequence encoding a second complementation pair member of a full-length selectable marker, wherein said second complementation pair member comprises a second amino acid mutation or modification that reduces selectable marker activity of the second complementation pair member such that selectable marker activity can be observed only in the presence of a first complementation pair member of the selectable marker which complements the second modification.

3. A second isolated nucleic acid molecule comprising
a sequence encoding a second polypeptide, wherein the second polypeptide is a subunit of a heteromeric protein, wherein the heteromeric protein is the heteromeric protein of claim 1, and
a sequence encoding a second complementation pair member of a full-length selectable marker, wherein the selectable marker is the selectable marker of claim 1, wherein said second complementation pair member of a selectable marker comprises a second amino acid mutation or modification that reduces selectable marker activity of the second complementation pair member such that selectable marker activity can be observed only in the presence of the first complementation pair member of claim 1.

4. An expression vector comprising the first nucleic acid of claim 1.

5. An expression vector comprising the second nucleic acid of claim 2.

6. The expression vector of claim 4 further comprising the second nucleic acid of claim 2.

7. An expression vector comprising the second nucleic acid of claim 3.

8. The nucleic acid of claim 1 wherein the first polypeptide comprises an antibody light chain or an antigen binding fragment thereof.

9. The nucleic acid of claim 2 wherein the second polypeptide comprises an antibody heavy chain or a antigen binding fragment thereof.

10. The isolated nucleic acid molecule of claim 1 or 2, wherein the selectable marker is selected from the group consisting of a drug resistance marker, a metabolic survival marker, a color marker and a fluorescent marker.

11. The isolated nucleic acid molecule of claims 1 or 2 wherein the complementation pair members are the same in the first nucleic acid and the second nucleic acid.

12. The isolated nucleic acid molecule of claim 11, wherein the selectable marker is selected from the group consisting of dihydrofolate reductase, neomycin resistance, hygromycin resistance, beta-galactosidase, and green fluorescent protein.

13. The nucleic acid of claim 12 wherein the selectable marker is a DHFR gene.

14. The nucleic acid of claim 13 wherein the first amino acid mutation or modification is in a Fragment 1, 2 subunit of the DHFR marker

15. The nucleic acid of claim 14 wherein the first amino acid mutation or modification is selected from the group consisting of a mutation at glutamic acid 30 of mouse DHFR and a rigid peptide linker inserted between Fragment 1, 2 and Fragment 3 of DHFR.

16. The nucleic acid of claim 15 wherein the first amino acid mutation or modification is Glu30Ala.

17. The nucleic acid of claim 15 wherein the first amino acid mutation or modification is a rigid peptide linker inserted between Fragment 1, 2 and Fragment 3 of DHFR.

18. The nucleic acid of 17 wherein the a rigid peptide linker is selected from the group consisting of an oligoproline sequence, an oligoglycine sequence, Gly-Gly-Pro repeats, GGGGS (SEQ ID NO: 1) repeats and the amino acid sequence PDALEAEIARLRKQIEALQGQNQHLQAAISQLKKVELFP (SEQ ID NO: 2).

19. The nucleic acid of claim 18 wherein the rigid peptide linker comprises the amino acid sequence GGPGGP.

20. The nucleic acid of claim 19 wherein the rigid peptide linker comprises the amino acid sequence PDALEAEIARLRKQIEALQGQNQHLQAAISQLKKVELFP (SEQ ID NO: 2).

21. The nucleic acid of claim 13 wherein the first amino acid mutation or modification is in a fragment 3 subunit of the DHFR marker

22. The nucleic acid of claim 21 wherein the first amino acid mutation or modification is a mutation at glycine 116 of mouse DHFR

23. The nucleic acid of claim 22 wherein the first amino acid mutation or modification is selected from the group consisting of Gly116Ala.

24. The nucleic acid of claim 13 wherein the second amino acid mutation or modification is in a fragment 1, 2 subunit of the DHFR marker

25. The nucleic acid of claim 24 wherein the second amino acid mutation or modification is selected from the group consisting of a mutation at glutamic acid 30 in mouse DHFR, and a rigid peptide linker inserted between Fragment 1, 2 and Fragment 3 of DHFR.

26. The nucleic acid of claim 25 wherein the second amino acid mutation or modification is Glu30Ala.

27. The nucleic acid of claim 25 wherein the second amino acid mutation or modification is a rigid peptide linker inserted between Fragment 1, 2 and Fragment 3 of DHFR.

28. The nucleic acid of claim 23 wherein the rigid peptide linker is selected from the group consisting of an oligoproline sequence, an oligoglycine sequence, Gly-Gly-Pro repeats, GGGGS (SEQ ID NO: 1) repeats and the amino acid sequence PDALEAEIARLRKQIEALQGQNQHLQAAISQLKKVELFP (SEQ ID NO: 2).

29. The nucleic acid of claim 28 wherein the rigid peptide linker comprises the amino acid sequence GGPGGP (SEQ ID NO: 3).

30. The nucleic acid of claim 28 wherein the rigid peptide linker is PDALEAEIARLRKQIEALQGQNQHLQAAISQLKKVELFP (SEQ ID NO: 2).

31. The nucleic acid of claim 13 wherein the second amino acid mutation or modification is in a Fragment 3 subunit of the DHFR marker

32. The nucleic acid of claim 31 wherein the second amino acid mutation or modification is a mutation at glycine 116 of mouse DHFR

33. The nucleic acid of claim 32 wherein the second amino acid mutation or modification is selected from the group consisting of Gly116Ala.

34. The isolated nucleic acid molecule of any one of claims 1, 2 or 3, wherein the first or second complementation pair member of a selectable marker is a fusion polypeptide comprising an interaction domain.

35. The isolated nucleic acid molecule of claim 34, wherein the interaction domain is a leucine zipper from a polypeptide selected from the group consisting of GCN4, C/EBP, c-Fos, c-Jun, c-Myc and c-Max

36. The isolated nucleic acid molecule of any one of claims 1, 2 or 3, further encoding a different functional selectable marker selected from the list consisting of zeomycin, neomycin, puromycin, Blasticidin S, and GPT.

37. A host cell comprising the isolated nucleic acid molecule of claims 1 or 2.

38. The host cell of claim 37 wherein the first and second nucleic acids are expressed on separate vectors.

39. The host cell of claim 37 wherein the first and second nucleic acids are expressed on the same vector.

40. The host cell of claim 37 which is selected from the group consisting of CHO, VERO, BHK, HeLa, Cos, MDCK, 293, 3T3, a myeloma cell line, and WI38 cells

41. A method of recombinantly expressing a heteromeric polypeptide comprising
culturing the host cell of claim 37 under conditions wherein the heteromeric protein is expressed.

42. The method of claim 41 wherein the heteromeric protein is an antibody.

43. The method of claim 41 further comprising isolating the heteromeric protein

44. The method of claim 41 wherein the host cell is selected from the group consisting of CHO, VERO, BHK, HeLa, Cos, MDCK, 293, 3T3, a myeloma cell line, and WI38 cells


SEQUENCE LISTING (3 sequences)

SEQ ID NO: 1 - DNA, 5 bp, Artificial sequence
  Synthetic primer
ggggs

SEQ ID NO: 2 - protein, 39 aa, Artificial sequence
  Synthetic primer
PDALEAEIARLRKQIEALQGQNQHLQAAISQLKKVELFP

SEQ ID NO: 3 - protein, 6 aa, Artificial sequence
  Synthetic peptide
GGPGGP